(19) United States
(12) Patent Application Publication
(10) Pub. No.: US 2025/0224393 A1
(43) Pub. Date: Jul. 10, 2025

(54) METHOD AND KIT FOR REDUCING INTERFERENCES IN IMMUNOASSAYS

(71) Applicant: Siemens Healthcare Diagnostics Products GmbH, Marburg (DE)

(72) Inventor: Juergen PATZKE, Marburg (DE)

(21) Appl. No.: 19/011,721

(22) Filed: Jan. 7, 2025

(30) Foreign Application Priority Data
Jan. 9, 2024 (EP) 24150860.5

(51) Int. Cl. G01N 33/53; G01N 33/543; G01N 33/68; G01N 33/86

(52) U.S. Cl. CPC G01N 33/5306; G01N 33/54306; G01N 33/68; G01N 33/86; G01N 2333/70596; G01N 2333/755

(57) ABSTRACT

A kit and method are described herein for detecting an analyte in a body fluid sample. The kit and method use a test-specific blocking antibody for reduction of interferences, for example as caused by heterophilic antibodies or rheumatoid factors.

### CROSS-REFERENCED TO RELATED PATENT APPLICATIONS

This patent application claims priority under 35 U.S.C. 119(a) to European patent application No. 24150860 filed on 9 Jan. 2024, which is hereby incorporated by reference in its entirety.

### REFERENCE TO AN ELECTRONIC SEQUENCE LISTING

This application contains references to amino acid sequences and/or nucleic acid sequences which have been submitted concurrently herewith as the sequence listing XML file entitled “32860HC-004083-US 17_Dec_2024_ST26.xml”, file size 4, 820 Bytes (B), created on 17 Dec. 2024. The aforementioned sequence listing is hereby incorporated by reference in its entirety.

### FIELD

This disclosure relates to the field of immunoassays for in vitro diagnostic applications and the use of a test-specific blocking antibody for reduction of interferences, as caused by heterophilic antibodies or rheumatoid factors.

### BACKGROUND

Immunoassays have been used for many decades in clinical diagnostic testing for the quantitative or qualitative detection of a variety of analytes in body fluid samples. The antibodies used for the direct detection of an antigen or for the indirect detection of a different analyte are typically monoclonal or polyclonal animal antibodies obtained from immunized animals (e.g., rabbit, mouse, sheep) or by biotechnological means. False test results caused by interfering substances from the patient sample can lead to far-reaching misdiagnoses. One problem that is known and occurs relatively frequently is that of interfering antibodies which may be intrinsically present in the sample of an individual (Bolstad, N. et al., Heterophilic antibody interference in immunometric assays. Best Practice & Research Clinical. Endocrinology & Metabolism (2013), 647-661). An example of interfering antibodies is heterophilic antibodies, i.e., antibodies in the patient blood that are directed against antigens, in particular immunoglobulins, of another species, for example human anti-mouse antibodies (HAMAS). Another example of interfering antibodies is so-called rheumatoid factors, i.e., human autoantibodies directed against the FC portion of human immunoglobulin G. All of these interfering antibodies typically have an affinity for animal antibodies and frequently bind to the Fc portion. If, then, a test system uses an animal antibody as part of the detection reaction for detection of an analyte (“analytical antibody”), what can occur in the presence of interfering antibodies from the patient sample is a binding reaction between analytical antibody and interfering antibodies that either blocks the detection reaction and causes a false-negative/falsely low result or boosts the detection reaction and causes a false-positive/falsely high result.

To reduce such interferences, it is therefore standard procedure to add blocking antibodies to modern immunoassays. Said blocking antibodies are typically mixtures of randomly selected nonspecific antibodies of the same immunoglobulin class and species as the analytical antibody used in the test system.

For example, what can be additionally added in a test system using a monoclonal mouse antibody (e.g., mouse IgG1) as analytical antibody is an excess of a mixture of irrelevant mouse IgG1, i.e., mouse IgG1 which is nonfunctional in the test system. In most cases, this causes any interfering antibodies from the patient sample to bind to the irrelevant antibodies, thereby preventing or at least minimizing the disruptive binding to the analytical antibody. Such blocking agents are commercially available, for example Heterophilic Blocking Reagent reagents (HBR) from Scantibodies Laboratory, Inc. or TRU Block reagents from Meridian Bioscience, Inc. The blocking effect of such irrelevant antibodies can be optimized by the prior aggregation of irrelevant antibodies (US 2004/0018556 A1).

Despite these measures, there are nevertheless cases in which certain samples cannot be analyzed correctly using a specific immunoassay test system owing to apparently insufficiently effective blocking by conventional blocking reagents. Bowyer A. E. et al. (Von Willebrand factor activity assay errors. Haemophilia (2016), 22, e74-e76) describe patient samples for which the use of two different test systems for determining von Willebrand factor (VWF) activity, involving the use of, inter alia, monoclonal mouse antibodies in both cases, repeatedly yielded falsely high results despite the addition of HAMA blockers (for blocking of human anti-mouse antibodies).

### SUMMARY

This section provides a general summary of the disclosure and is not a comprehensive disclosure of its full scope or all of its features.

In one embodiment, a kit for use in a method for detecting an analyte in a body fluid sample is provided herein. The kit may comprise:
a) a first antigen-specific antibody which has a first amino acid sequence, which binds specifically to an antigen, and the use of which in a defined test system for detection of the analyte brings about an analyte-specific detection reaction, and
b) an antibody variant which has a second amino acid sequence and the antigen-binding capacity of which is greatly reduced compared to the first antibody such that its additional use in the defined test system for detection of the analyte reduces the analyte-specific detection reaction by a maximum of 15%,
   wherein the amino acid sequence of the antibody variant, with the exception of one to three modified amino acid residues, is identical to the amino acid sequence of the first antigen-specific antibody.

In another embodiment, a method for detecting an analyte in a body fluid sample is provided herein. The method may comprise:
a) providing a reaction mixture by mixing the sample with
   i. a first antigen-specific antibody which has a first amino acid sequence, which binds specifically to an antigen, and the use of which in a defined test system for detection of the analyte brings about an analyte-specific detection reaction, and
   ii. an antibody variant which has a second amino acid sequence and the antigen-binding capacity of which is greatly reduced compared to the first antibody such that its additional use in the defined test system for detection of the analyte reduces the analyte-specific detection reaction by a maximum of 15%; and
b) measuring a measurement variable in the reaction mixture, the measurement variable being influenced by the formation of a complex of antigen and the first antigen-specific antibody and correlating with the amount of analyte,
   wherein the amino acid sequence of the antibody variant, with the exception of one to three modified amino acid residues, is identical to the amino acid sequence of the first antigen-specific antibody.

### BRIEF DESCRIPTION OF FIGURES

The drawings described herein are for illustrative purposes only of selected embodiments and not all possible implementations and are not intended to limit the scope of the present disclosure.

FIG. 1A shows a chart showing the VWF activities [% of the norm], measured in a normal sample (1) and in a sample having reduced VWF activity (2), in the absence and in the presence of the novel antibody variant at different concentrations in the reaction mixture (mg/mL).

FIG. 1B shows a chart showing the VWF activities [% of the norm], measured in a normal sample (3) and in a sample having reduced VWF activity (4), using different dilutions of the commercial HBR-1 reagent (mg/mL total protein content in the reaction mixture).

FIG. 2 shows a chart showing the VWF activities [% of the norm], measured in a sample containing HAMA antibody, with additional use of different dilutions of the commercial HBR-1 reagent (mg/mL total protein content in the reaction mixture) (1), with additional use of different concentrations of the novel antibody variant in the reaction mixture (mg/mL) (2) or with additional use of different concentrations of the novel antibody variant in the reaction mixture (mg/mL) in combination with the HBR-1 reagent (3).

### DETAILED DESCRIPTION

An “immunoassay” in the context of the present disclosure is a method for detecting an analyte in a sample that comprises the use of at least one antigen-specific antibody. The antigen-specific antibody may be, but need not necessarily be, an analyte-specific antibody.

Depending on the test setup, the antibody used may perform a variety of functions. For example, it may be used as capture antibody or as labelled secondary antibody for direct binding and detection of the analyte; to this end, the antibody must be an analyte-specific antibody. In another case, the antibody may be used, for example, to immobilize on a solid phase a binding partner of the analyte to be detected; to this end, the antibody must be an antibody having specificity for said binding partner. A variety of immunoassay principles are known (direct, indirect, competitive, noncompetitive). Common to all is that they comprise the use of at least one antigen-specific antibody which, in the chosen test system for detection of the analyte, is directly or indirectly involved in the analyte-specific detection reaction.

Conventional immunoassays optimized to minimize the occurrence of antibody-induced interferences are thus methods for detecting an analyte in a body fluid sample that essentially comprise the steps of:
i) providing a reaction mixture by contacting the sample with
   a) an antigen-specific antibody which binds specifically to the antigen and
   b) a mixture of nonspecific, randomly selected antibodies which are known from experience to block the binding of interfering antibodies present in the sample to the antigen-specific antibody, and
ii) measuring a measurement variable in the reaction mixture, the measurement variable being influenced by the formation of a complex of antigen and the first, antigen-specific correlating with the amount of analyte.

Since there are always samples which, despite the addition of blocking antibodies to the reaction mixture, cannot be analyzed correctly in certain immunoassays owing to apparently insufficiently effective blocking, it is an object of the disclosure to provide further methods and means for immunoassays that increase the reliability of immunoassays by effectively reducing any interferences caused by interfering antibodies, such as heterophilic antibodies or rheumatoid factors, present in a patient sample.

The object is achieved according to one embodiment by using a blocking antibody which is derived from the analytical antibody and which has a structure virtually identical to that of the analytical antibody. As a result of this, any interfering antibodies from the patient sample that apparently bind highly specifically to an epitope of the analytical antibody and are therefore not blocked or not sufficiently blocked by the classic, randomly selected blocking antibody mixtures are now specifically caught and thus blocked by binding to the derived blocking antibody. It has been found that an antibody having an amino acid sequence which, with the exception of one to three modified amino acid residues, is identical to the amino acid sequence of the analytical antibody, thereby greatly reducing its antigen-binding capacity compared to the analytical antibody, brings about efficient blocking of interfering antibodies and thus significantly improves the reliability of the immunoassay.

### Kit

In one embodiment, the present disclosure therefore provides a kit for use in a method for detecting an analyte in a body fluid sample. The kit may contain:
i) a first antigen-specific antibody which has a first amino acid sequence, which binds specifically to an antigen, and the use of which in a defined test system for detection of the analyte brings about an analyte-specific detection reaction (“analytical antibody”), and
ii) an antibody variant which has a second amino acid sequence, wherein the antigen-binding capacity of the antibody variant is greatly reduced compared to the first antibody such that, or the competition thereof with the first antigen-specific antibody for binding to the antigen is so low that, its additional use in the defined test system for detection of the analyte reduces the analyte-specific detection reaction by a maximum of 15% (also called “nonanalytical antibody” or “nonanalytical antibody variant”),
   and wherein the amino acid sequence of the antibody variant, with the exception of one to three modified amino acid residues, is identical to the amino acid sequence of the first antigen-specific antibody.

A kit for use in a method for detecting an analyte in a body fluid sample typically comprises one or more reagents in liquid or lyophilized form or in the form of coated solid phases that are contacted with the body fluid sample to be analyzed (e.g., whole blood, plasma, serum, urine) in order to bring about a reaction detection allowing quantitative, semiquantitative or qualitative determination of the amount or activity of the analyte.

The first antigen-specific antibody binds specifically to an antigen and is essential for generating the intended analyte-specific detection reaction of the test system (“analytical antibody”). The antigen-specific antibody may be an analyte-specific antibody which binds specifically to an analyte from the body fluid sample. Alternatively, the antibody may be one which binds specifically to a binding partner of the analyte. In this case, the binding partner of the analyte may be one which is intrinsically present in the body fluid sample or which is added to the reaction mixture. In another embodiment of a detection method, the antibody may be one which binds specifically to a cleavage product of the analyte.

Depending on the test setup, the antibody used may perform a variety of functions. For example, it may be used as capture antibody or as labelled secondary antibody for direct binding and detection of the analyte; to this end, the antibody must be an analyte-specific antibody. In another case, the antibody may be used, for example, to immobilize on a solid phase a binding partner of the analyte to be detected; to this end, the antibody must be an antibody having specificity for said binding partner. A variety of immunoassay principles are known (direct, indirect, competitive, noncompetitive). Common to all is that they comprise the use of at least one antigen-specific antibody which, in the chosen test system for detection of the analyte, is directly or indirectly involved in the analyte-specific detection reaction.

The first antigen-specific antibody may belong to any immunoglobulin class (IgA, IgD, IgE, IgG or IgM); the original source thereof may be human, mouse, rabbit, mouse, sheep, camel or some other animal. Preferably, the antibody is a monoclonal antibody or a recombinantly produced antibody. The first antigen-specific antibody may also be a chimeric antibody or a humanized antibody. The term “first antigen-specific antibody” expressly includes not only complete antibodies, but also various antigen-binding antibody fragments, for example Fab or F(ab)₂ fragments.

In various embodiments, the first antigen-specific antibody may be associated with a solid phase and/or a component of a signal-forming system.

The term “solid phase” in the context of this invention includes an object which consists of porous and/or nonporous, water-insoluble material and which can take a variety of forms, for example a vessel, a small tube, a microtitration plate (ELISA plate), beads, microparticles, rods, strips, filter or chromatography paper, etc. Generally, the surface of the solid phase is hydrophilic or can be rendered hydrophilic. The solid phase can consist of a variety of materials, for example inorganic and/or organic materials, synthetic materials, naturally occurring materials and/or modified naturally occurring materials. Examples of solid-phase materials are polymers, for example cellulose, nitrocellulose, cellulose acetate, polyvinyl chloride, polyacrylamide, crosslinked dextran molecules, agarose, polystyrene, polyethylene, polypropylene, polymethacrylate or nylon; latex; ceramic; glass; metals, in particular precious metals such as gold and silver; magnetite; mixtures or combinations of same. Particles, including magnetic particles and latex particles, can be labeled with dyes, sensitizers, fluorescent substances, chemiluminescent substances, isotopes or other detectable labels.

A “component of a signal-forming system” is a molecule which itself produces a signal or can induce the production of a signal, for example a fluorescent substance, a chemiluminescent substance, a radioactive substance or an enzyme. The signal can be detected or measured, for example, on the basis of enzyme activity, luminescence, light absorption, light scattering, emitted electromagnetic or radioactive radiation or a chemical reaction.

Suitable components of a signal-forming system are, for example, enzymes, including horseradish peroxidase, alkaline phosphatase, glucose-6-phosphate dehydrogenase, alcohol dehydrogenase, glucose oxidase, β-galactosidase, luciferase, urease and acetylcholinesterase; enzyme substrates; dyes; fluorescent substances, including fluorescein isothiocyanate, rhodamine, phycoerythrin, phycocyanin, ethidium bromide, 5-dimethylaminonaphthalene-1-sulfonyl chloride and fluorescent chelates of rare earths; chemiluminescent substances including luminol, isoluminol, acridinium compounds, olefin, enol ethers, enamine, aryl vinyl ethers, dioxene, arylimidazole, lucigenin, luciferin and aequorin; sensitizers including eosin, 9,10-dibromoanthracene, methylene blue, porphyrin, phthalocyanine, chlorophyll, rose bengal; coenzymes; radioactive isotopes including ¹²⁵I, ¹³¹I, ¹⁴C, ³H, ³²P, ³³P, ³⁵S, ⁵¹Cr, ⁵⁹Fe, ⁵⁷Co and ⁷⁵Se.

The term “associated” is to be understood broadly and includes, for example, a covalent bond and a noncovalent bond, direct binding and indirect binding, adsorption to a surface, and inclusion in a depression. In the case of a covalent bond, the first antigen-specific antibody or antigen-specific antibody fragment is bound to a solid phase or to a component of a signal-forming system via a chemical bond. An example of a noncovalent bond is surface adsorption. Besides direct binding, the first antigen-specific antibody or antigen-specific antibody fragment may also be indirectly bound to the solid phase via specific interaction with other binding partners, for example via specific interaction with avidin if the first antigen-specific antibody or antigen-specific antibody fragment is biotinylated.

The antibody variant of the kit according to the invention is an antibody derived from the first antigen-specific antibody or a corresponding antibody fragment (also called “nonanalytical antibody” or “nonanalytical antibody variant”) and has 1.) an amino acid sequence which, with the exception of one to three modified amino acid residues, is identical to the amino acid sequence of the first antigen-specific antibody and 2.) an antigen-binding capacity which is greatly reduced compared to the first antigen-specific antibody such that its additional use in the defined test system for detection of the analyte reduces the analyte-specific detection reaction by a maximum of 15%.

Such a “nonanalytical” antibody variant is thus individually tailored to the first antigen-specific antibody (“analytical antibody”) and is typically obtainable by defining a modified amino acid sequence from the known amino acid sequence of the analytical antibody (or after the amino acid sequence of the analytical antibody has been determined) through the substitution, deletion, insertion or chemical derivatization of one to three amino acid residues, and recombinantly producing a correspondingly modified antibody variant. The position of the one modified amino acid residue or the positions of the two or three modified amino acid residues are to be chosen such that they are in a region of the analytical antibody which is relevant to antigen binding and which is to be functionally restricted or deactivated by the modification of the amino acid sequence, such that the antigen-binding capacity of the resultant modified nonanalytical antibody variant is absent or at least only greatly reduced. For this purpose, the one to three amino acid residues are preferably modified, i.e., substituted, deleted, inserted or chemically derivatized, in one or more of the complementarity-determining regions (CDRs) of the heavy or light chain of the analytical antibody. The complementarity-determining regions (CDRs) of the heavy chain of an antibody (CDR-H1, CDR-H2 and CDR-H3) and the light chain of an antibody (CDR-L1, CDR-L2 (according to the Kabat numbering scheme), which are separated from each other by so-called framework regions, are well known to a person skilled in the art. Particularly preferably, at least one amino acid residue in a complementarity-determining region of the heavy chain of the analytical antibody is modified. More preferably, at least one amino acid residue in the complementarity-determining region CDR-H3 of the heavy chain of the analytical antibody is modified.

In one embodiment of the test kit, there is thus an antibody variant in which the one to three modified amino acid residues are located in one or more of the complementarity-determining regions (CDRs) of the heavy or light chain of the antibody variant.

In a further embodiment of the test kit, there is an antibody variant in which at least one modified amino acid residue is located in one complementarity-determining region of the heavy chain of the antibody variant.

In yet a further embodiment of the test kit, there is an antibody variant in which at least one modified amino acid residue is located in the complementarity-determining region CDR-H3 of the heavy chain of the antibody variant.

A “modified amino acid residue” is to be understood to mean an amino acid residue which, in relation to a position in the succession of amino acids of the primary sequence of the analytical antibody, has been substituted, deleted, inserted or chemically derivatized. In the case of a substitution, the original amino acid residue is replaced by a different amino acid residue. Preferably, the substitutions are nonconservative substitutions, i.e., substitutions between different families of amino acids differing in their side chains and chemical properties. Examples of different families are amino acids having basic side chains, having acidic side chains, having nonpolar aliphatic side chains, having nonpolar aromatic side chains, having polar side chains, having uncharged polar side chains, having charged side chains, having small side chains, having large side chains, etc. For example, a small amino acid residue is replaced by a large amino acid residue or a charged amino acid residue is replaced by an uncharged amino acid residue.

The “nonanalytical” antibody variant may also be a naturally occurring variant of the first antigen-specific antibody, wherein the amino acid sequence of the variant has one to three substituted, deleted or inserted amino acid residues compared to the original analytical antibody.

The antibody variant is thus, compared to the first antigen-specific antibody, a nonfunctional or at least less functional variant thereof in relation to antigen-binding capacity.

The antigen-binding capacity of the antibody variant must be greatly reduced compared to the first antigen-specific antibody such that its additional use in the defined test system for detection of the analyte reduces the analyte-specific detection reaction by a maximum of 15%.

A reduced antigen-binding capacity of the antibody variant can be measured in a comparative experiment with the first antigen-specific antibody in a standard assay for determining the specificity of binding of the target antigen, for example in an ELISA assay, a BIAcore™ assay, a Octet® BLI assay, or a FACS-based assay if the antigen is expressed on a cell surface.

However, it is crucial that the antigen-binding capacity of the antibody variant is tested in the same defined test system for detection of the analyte in which the first antigen-specific antibody is used as “analytical” antibody. The term “defined test system” refers to a test setup defined with respect to the components used and the method steps. Variation of a single component or a single method step in the otherwise unchanged test setup makes it possible to determine the influence of said variation in the otherwise defined test system. Ideally, the method for detecting an analyte is used as a defined test system, wherein the use of the first antigen-specific antibody and the antibody variant is intended for realization thereof. In relation to the invention, what are used to this end are the first antigen-specific antibody for the purpose of detection of the analyte and additionally, i.e., in combination with the first antigen-specific antibody, the antibody variant to be tested.

In the test system in which the first antigen-specific antibody is used as an “analytical” antibody, a suitable antibody variant does not have a significant competitive effect on the analyte-specific detection reaction. This is ensured by first measuring in the test system over the entire measurement range the reaction intensity of the analyte-specific detection reaction with and without addition of the antibody variant to the reaction mixture in samples without interfering antibodies. A suitable antibody variant is an antibody variant having an antigen-binding capacity which is greatly reduced compared to the first antigen-specific antibody such that its presence does not reduce the reaction intensity of the analyte-specific detection reaction by more than 15%, preferably by more than 10% and particularly preferably by more than 5%; the reduced antigen-binding capacity of the antibody variant is functionally demonstrated by the little or no competition between the antibody variant and the first antigen-specific antibody for binding to the antigen, i.e., the little or no competition for the binding site(s) of the antigen, by showing that the use of the antibody variant in combination with the first antigen-specific antibody does not have an excessively disruptive influence on the analyte-specific detection reaction, but instead brings about a maximum reduction in the reaction intensity of the analyte-specific detection reaction of 15%.

In one embodiment of the kit, the first antigen-specific antibody and the antibody variant may be present in different reagents or in a single reagent.

In one embodiment of the kit, the first antigen-specific antibody is associated with a solid phase, for example on the surface of a vessel (as described above), for example on the bottom of a well of a microtiter plate or on the inside of a reaction tube. Such a kit is particularly suitable for carrying out heterogeneous test methods, for example ELISA tests. Such a kit preferably further contains a further vessel containing the antibody variant, preferably as a constituent of a liquid reagent (or of a lyophilizate thereof).

In another embodiment of the kit, the first antigen-specific antibody is associated with a surface of a particulate solid phase (as described above). To this end, the kit contains a vessel containing the corresponding reagent in the form of a liquid suspension or a resuspendable lyophilizate thereof. Such a test kit is suitable for measuring agglutination by means of photometric methods.

In yet another embodiment of the kit, the first antigen-specific antibody is associated with a component of a signal-forming system (as described above). The first antigen-specific antibody may be directly associated with a component of a signal-forming system or indirectly associated, for example when the antibody and the component of a signal-forming system are associated with a single solid phase, for example with a latex particle. To this end, the kit contains a vessel containing the corresponding reagent in liquid form or as resuspendable lyophilizate thereof. Depending on the nature of the signal-forming system, such a test kit is suitable, for example, for measurements of chemiluminescence, fluorescence or change in absorption.

In a particular embodiment of the kit, the first antigen-specific antibody is an analyte-specific antibody. In this case, the antibody is used directly as capture antibody or as labelled secondary antibody for binding and detection of the analyte, for example in a sandwich immunoassay.

In another embodiment of the kit, present besides the first antigen-specific antibody is additionally a different, second antigen-specific antibody, for example in a kit for use in a sandwich immunoassay, wherein the second antigen-specific antibody may be specific for the same antigen as the first antigen-specific antibody or specific for a different antigen. In such a kit, preferably present is a further “nonanalytical” antibody variant (as described above) which, compared to the second antigen-specific antibody, is a nonfunctional or at least less functional variant thereof in relation to antigen-binding capacity. Such a kit thus additionally may contain:
c) a second antigen-specific antibody which has a third amino acid sequence, which binds specifically to an antigen, and the use of which in a defined test system for detection of the analyte brings about an analyte-specific detection reaction, and
d) a further antibody variant which has a fourth amino acid sequence and the antigen-binding capacity of which is greatly reduced compared to the second antigen-specific antibody such that its additional use in the defined test system for detection of the analyte reduces the analyte-specific detection reaction by a maximum of 15%,
   and wherein the amino acid sequence of the further antibody variant, with the exception of one to three modified amino acid residues, is identical to the amino acid sequence of the second antigen-specific antibody.

In a further particular embodiment of the kit, each antigen-specific antibody present is provided with an antibody variant which, compared to the respective antigen-specific antibody, is a nonfunctional or at least less functional variant thereof in relation to antigen-binding capacity (as described above).

### Use

In another embodiment, this disclosure further provides for the use of a kit in a method for detecting an analyte in a body fluid sample.

Particular preference is given to the use of a kit according to the invention for interference-free detection of an analyte in a body fluid sample containing interfering antibodies, for example from the group of heterophilic antibodies and autoantibodies.

In a specific embodiment of the kit, the first antigen-specific antibody is an antibody which binds specifically to glycoprotein Ib (GPIb) protein. GPIb protein is a binding partner of von Willebrand factor (VWF) and is used in various assays for determination of VWF activity (see, for example, WO 2009/007051 A2). Qualitative defects or functional defects of VWF are detected through reduced binding of the VWF present in the sample to the GPIb protein added. The capacity of VWF to bind to added GPIb protein may be quantitatively determined by configuring the test methods such that the formation of a complex between VWF and GPIb in the test reaction can be measured, for example by measuring the agglutination of latex particles which have been coated with an anti-GPIb antibody and which only agglutinate if VWF-GPIb complexes are formed in the test reaction and are in turn then bound by the latex particle-associated anti-GPIb antibodies. As mentioned above, it has been observed that such an assay using a mouse monoclonal anti-GPIb antibody repeatedly yields falsely high results despite the addition of HAMA blockers (for blocking of human anti-mouse antibodies).

In a particular form of the specific embodiment of the kit,
the first antigen-specific antibody is an antibody which binds specifically to glycoprotein Ib (GPIb) protein and which has, in the complementarity-determining region CDR-H3 of the heavy chain (according to the Kabat numbering scheme), the amino acid sequence according to SEQ ID NO. 1 (DTMIKGHYVMDY), and
the antibody variant is an antibody, the amino acid sequence of which, with the exception of two modified amino acid residues, is identical to the amino acid sequence of the first GPIb protein-specific antibody and which has, in the complementarity-determining region CDR-H3 of the heavy chain (according to the Kabat numbering scheme), the amino acid sequence according to SEQ ID NO. 2 (DTMIKGHSVEDY).

Such a test kit is suitable for use in a method for detecting the VWF activity in a body fluid sample and has the particular advantage of allowing the interference-free detection of the VWF activity in a body fluid sample containing interfering antibodies, for example from the group of heterophilic antibodies and autoantibodies.

### Method

In another embodiment, this disclosure further provides a method for detecting an analyte in a body fluid sample, the method comprising the steps of:
a) providing a reaction mixture by mixing the sample with
   i. a first antigen-specific antibody which has a first amino acid sequence, which binds specifically to an antigen, and the use of which in a defined test system for detection of the analyte brings about an analyte-specific detection reaction, and
   ii. an antibody variant which has a second amino acid sequence and the antigen-binding capacity of which is greatly reduced compared to the first antibody such that its additional use in the defined test system for detection of the analyte reduces the analyte-specific detection reaction by a maximum of 15%; and
b) measuring a measurement variable in the reaction mixture, the measurement variable being influenced by the formation of a complex of antigen and the first antigen-specific antibody and correlating with the amount of analyte,
   wherein, here too (as already described above), the amino acid sequence of the antibody variant, with the exception of one to three modified amino acid residues, is identical to the amino acid sequence of the first antigen-specific antibody.

In a particular embodiment of the method described herein, the sample is first mixed with the antibody variant and the mixture thus produced is incubated, and only then is the first antigen-specific antibody added to the mixture. This preincubation of the sample with the “nonanalytical” antibody variant brings about particularly efficient blocking of interfering antibodies, since they are already bound before they come into contact with the “analytical” antibody.

In one embodiment of the method described herein, the first antigen-specific antibody is an analyte-specific antibody and the measurement variable measured is influenced by the formation of a complex of analyte and the first analyte-specific antibody. An example thereof is an immunoassay in which a complex of analyte and a latex particle-associated analyte-specific antibody is formed and the formation of the complex is determined photometrically on the basis of the agglutination reaction of the latex particles in the reaction mixture.

In another embodiment of the method described herein, the first antigen-specific antibody is an antibody having specificity for a binding partner of the analyte, and the measurement variable measured is influenced by the formation of a complex of analyte and the binding partner of the analyte and the first antigen-specific antibody having specificity for the binding partner of the analyte. An example thereof is a functional binding test by means of which it is not the amount of an analyte but rather the capacity thereof to bind to a specific binding partner that is to be measured and in which a complex of analyte, binding partner of the analyte and a, for example, latex particle-associated antibody having specificity for the binding partner is formed and the formation of the complex is determined photometrically on the basis of the agglutination reaction of the latex particles in the reaction mixture.

In a specific embodiment, a method for detecting the activity of von Willebrand factor in a body fluid sample is provided herein, wherein the first antigen-specific antibody is an antibody having specificity for GPIb protein, and wherein the measurement variable measured is influenced by the formation of a complex of von Willebrand factor and the GPIb protein and the first antigen-specific antibody having specificity for GPIb protein.

In one embodiment of the method described herein, the first antigen-specific antibody may be associated with a particulate solid phase and the agglutination of the particulate solid phase in the reaction mixture, which is influenced by the formation of a complex of antigen and the first antigen-specific antibody and which correlates with the amount of analyte, may be measured.

The agglutination of the particulate solid phase in the reaction mixture can be measured photometrically, for example turbidimetrically or nephelometrically. Binding tests based on the principle of particle-enhanced light scattering have been known since about 1920 (for a review, see Newman, D. J. et al., Particle enhanced light scattering immunoassay. Ann Clin Biochem 1992; 29:22-42). Preference is, in this context, given to polystyrene particles having a diameter of 0.1 to 0.5 μm, more preferably having a diameter of 0.15 to 0.35 μm. Preference is given to using polystyrene particles having amine, carboxyl or aldehyde functions. Preference is also given to using core-and-shell particles. The synthesis of the particles and the covalent coupling of ligands is described for example in Peula, J. M. et al., Covalent coupling of antibodies to aldehyde groups on polymer carriers. Journal of Materials Science: Materials in Medicine 1995; 6:779-785.

Alternatively, the agglutination of the particulate solid phase in the reaction mixture can be measured by measuring a signal generated by a signal-forming system when the first and the second component of the signal-forming system are brought into spatial proximity. In this context, a first fraction of the particulate solid phase is associated with a first component of a signal-forming system and a second fraction of the particulate solid phase is associated with a second component of the signal-forming system, and the first and the second component of the signal-forming system cooperate such that a detectable signal is produced when the first and the second component of the signal-forming system are brought into spatial proximity and the agglutination of the particulate solid phases in the reaction mixture is measured on the basis of the signal produced.

In this embodiment of the method described herein, the signal-forming system comprises at least a first and a second component, which cooperate such that a detectable signal is produced when they are brought into spatial proximity and are thereby able to interact with one another. An interaction between the components is to be understood as meaning in particular an energy transfer, i.e. the direct transfer of energy between the components, for example through irradiation with light or electrons or via reactive chemical molecules such as short-lived singlet oxygen. The energy transfer may be from one component to the other, but a cascade of different substances via which the energy transfer proceeds is also possible. For example, the components may be a pair comprising an energy donor and an energy acceptor, for example a photosensitizer and a chemiluminescent agent (EP-A2-0515194, LOCI® Technologie) or photosensitizer and fluorophore (WO 95/06877) or radioactive iodine-125 and fluorophore (Udenfriend et al. (1985) Proc. Natl. Acad. Sci. 82:8672-8676) or fluorophore and fluorescence quencher (U.S. Pat. No. 3,996,345). Particularly preferably, the first component of the signal-forming system is a chemiluminescent agent and the second component of the signal-forming system is a photosensitizer or vice-versa, and it is the chemiluminescence in the reaction mixture that is measured.

The following examples illustrate the invention described herein and are not to be understood as limiting.

### EXAMPLES

### Example 1: Latex Agglutination Assay for Determination of VWF Activity According to the Prior Art

### Reagent 1:

HBR-1 reagent (Heterophilic Blocking Reagent 1, Scantibodies Laboratory, Inc., Santee, USA) containing a mixture of mouse immunoglobulins for binding to heterophilic antibodies

### Reagent 2:

Recombinantly expressed GPIb protein fragment of a gain-of-function mutant of the human GPIb protein in buffer solution.

### Reagent 3:

Suspension of polystyrene particles (latex particles) coated with a mouse monoclonal anti-GPIb antibody.

The von Willebrand factor (VWF) activity in a plasma sample is determined as follows:
1. 40 μL of sample are mixed with 2 μL of reagent 1 and the sample thus treated is incubated at room temperature for 30 minutes.
2. 15 μL of the pretreated sample are then mixed with 30 μL of Owren's Veronal buffer and with 70 μL of a further detergent-containing buffer and with 15 μL of reagent 2, and the mixture is incubated at 37° C. for 2 minutes.
3. 40 μL of reagent 3 are then added to the mixture, and the change in absorbance of the reaction mixture is measured with light of a wavelength of 570 nm.
4. The raw values measured are evaluated with the aid of a calibration curve.

Despite the use of the HBR-1 reagent, there are occasionally samples which yield falsely high results owing to the apparently insufficiently effective blocking of HAMA that is intended by reagent 1.

### Example 2: Production of a Variant of the Anti-GPIB Antibody Having Reduced GPIb-Binding Capacity

The complete amino acid sequence of the mouse monoclonal anti-GPIb antibody used as an analytical antibody in the VWF assay according to Example 1 was determined.

The amino acid sequence in the region of the heavy, variable chain of said anti-GPIb antibody comprising the complementarity-determining regions [symbol] , CDR-H2 and CDR-H3 (according to the Kabat numbering scheme) was as follows:

(SEQ ID NO. 3)

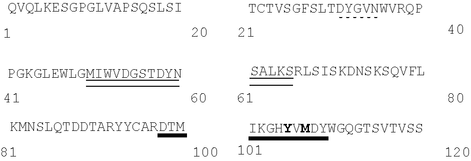

A variant of said anti-GPIb antibody having reduced GPIb-binding capacity was produced by substitution, in the CDR-H3 region, of the amino acid residues at positions 105 and 107 Of SEQ ID NO. 3. At position 105, the relatively large amino acid tyrosine (Y) was replaced by the very small and short amino acid serine(S) (in bold in the two sequences shown). At position 107, methionine (M) was replaced by phenylalanine (F), which corresponds to a reversion to the mouse antibody germ line (in bold in the two sequences shown). An appropriately coding nucleic acid molecule was derived, and standard genetic engineering methods were used to establish a transgenic expression cell line expressing the modified antibody, the amino acid sequence of which, with the exception of the two aforementioned modified amino acid residues, is identical to the amino acid sequence of the anti-GPIb antibody.

The amino acid sequence in the region of the heavy, variable chain of said modified antibody comprising the complementarity-determining regions [symbol] , CDR-H2 and CDR-H3 was thus as follows:

(SEQ ID NO. 4)

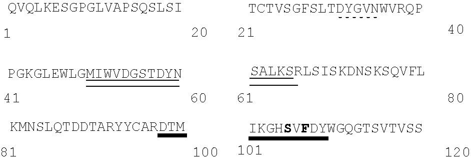

### Example 3: Detection of the Reduced GPIb-Binding Capacity of the Novel Antibody Variant

The test system used was the latex agglutination assay for determination of VWF activity according to Example 1.

The assay was modified such that, instead of reagent 1 (HBR-1 reagent), 2 μL of reagents containing different amounts of the novel antibody variant produced according to Example 2 were, in each case, mixed and incubated with 40 μL of a normal plasma sample or a sample known to have reduced VWF activity.

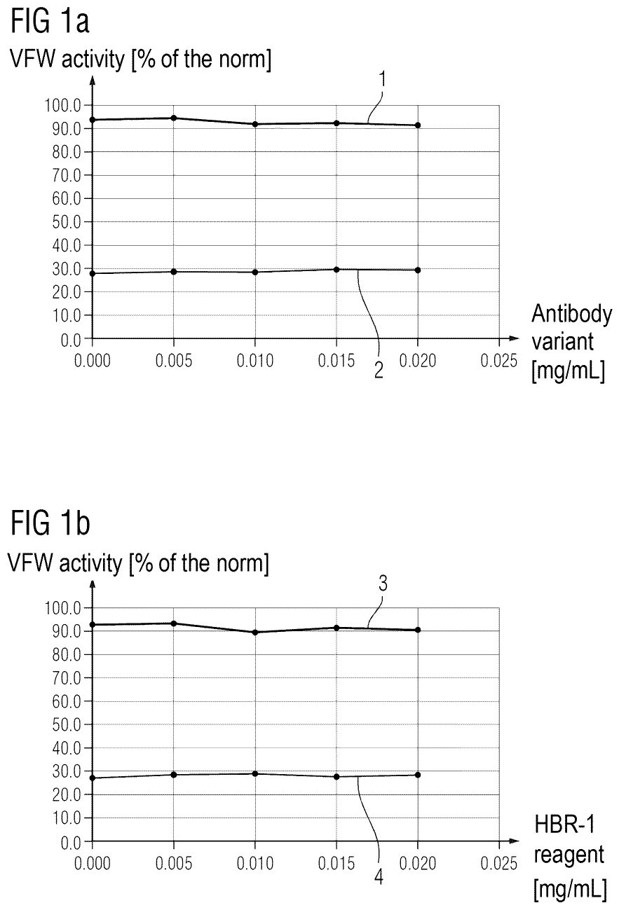

The results are shown in FIG. 1A. At final concentrations of up to 0.02 mg/mL of the novel antibody variant in the final reaction mixture, the VWF activity measured is reduced by a maximum of 2.3% compared to reaction mixtures to which the novel antibody variant was not added (0 mg/mL). This shows that the novel antibody variant does not compete with the functional (“analytical”) mouse monoclonal anti-GPIb antibody for the binding site on the GPIb protein.

The novel antibody variant produced according to Example 2, the amino acid sequence of which, with the exception of the two aforementioned modified amino acid residues, is identical to the amino acid sequence of the functional anti-GPIb antibody, thus has a greatly reduced GPIb-binding capacity compared to the functional anti-GPIb antibody.

For the purposes of comparison, the assay was modified in a further variant such that 2 μL of different dilutions of reagent 1 (HBR-1 reagent) containing different total protein concentrations were, in each case, mixed and incubated with 40 μL of a normal plasma sample or a sample known to have reduced VWF activity.

The results are shown in FIG. 1B. At final concentrations of up to 0.02 mg/mL of HBR total protein in the final reaction mixture, the VWF activity measured is reduced by a maximum of 2.6% compared to reaction mixtures to which HBR-1 reagent was not added (0 mg/mL). This observation supports the conclusion that the reduction in VWF activity caused by the novel antibody variant is not a specific effect of the antibody variant.

### Example 4: Detection of the HAMA Antibody-Blocking Effect of the Novel Antibody Variant

The latex agglutination assay for determination of VWF activity according to Example 1 was modified such that, instead of reagent 1 (HBR-1 reagent), 2 μL of reagents containing different amounts of the novel antibody variant produced according to Example 2 alone or containing different amounts of the novel antibody variant produced according to Example 2 in combination with reagent 1 (HBR-1 reagent) were, in each case, mixed and incubated with 40 μL of a HAMA antibody-containing plasma sample having known VWF activity. The sample used was distinguished by the fact that it could not be sufficiently blocked by the sole use of HBR-1 reagent and falsely high VWF activity was therefore determined.

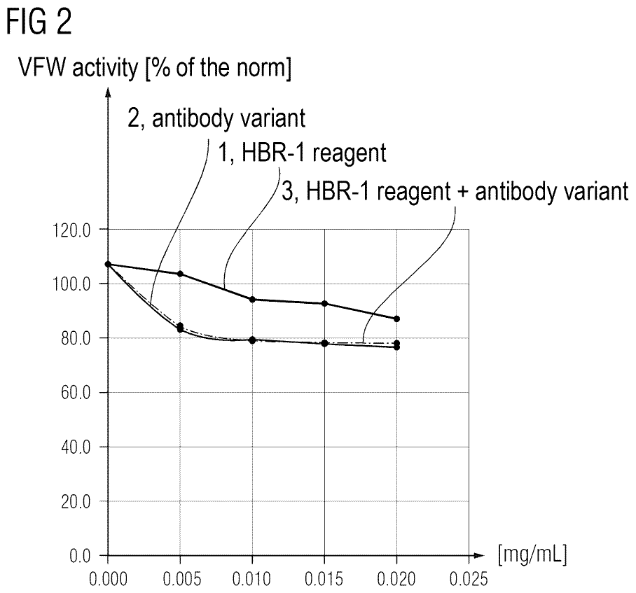

The results are shown in FIG. 2. At final concentrations of from 0.005 mg/mL of the novel antibody variant in the final reaction mixture, what can already be observed is virtually complete blocking of the interfering effect of the HAMA antibodies. By contrast, the sole use of HBR-1 reagent brings about only insufficient blocking of the interfering effect of the HAMA antibodies. The combination of the novel antibody variant with the HBR-1 reagent did not show a blocking effect that went beyond the blocking effect of the novel antibody variant or that indicated it was impaired.

What is claimed is:

1. A kit for use in a method for detecting an analyte in a body fluid sample, the kit comprising:
a) a first antigen-specific antibody which has a first amino acid sequence, which binds specifically to an antigen, and the use of which in a defined test system for detection of the analyte brings about an analyte-specific detection reaction, and
b) an antibody variant which has a second amino acid sequence and the antigen-binding capacity of which is greatly reduced compared to the first antibody such that its additional use in the defined test system for detection of the analyte reduces the analyte-specific detection reaction by a maximum of 15%,
wherein the amino acid sequence of the antibody variant, with the exception of one to three modified amino acid residues, is identical to the amino acid sequence of the first antigen-specific antibody.

2. The kit as claimed in claim 1, wherein the one to three modified amino acid residues are located in one or more of the complementarity-determining regions (CDRs) of the heavy or light chain of the antibody variant.

3. The kit as claimed in claim 2, wherein at least one modified amino acid residue is located in one complementarity-determining region of the heavy chain of the antibody variant.

4. The kit as claimed in claim 3, wherein at least one modified amino acid residue is located in the complementarity-determining region CDR-H3 of the heavy chain of the antibody variant.

5. The kit as claimed in claim 1, wherein the antibody variant is produced recombinantly.

6. The kit as claimed in claim 1, wherein the first antigen-specific antibody and the antibody variant are present in different reagents.

7. The kit as claimed in claim 1, wherein the first antigen-specific antibody is associated with a solid phase and/or a component of a signal-forming system.

8. The kit as claimed in claim 1, wherein the first antigen-specific antibody is an analyte-specific antibody.

9. The kit as claimed in claim 1, further comprising:
c) at least one further antigen-specific antibody which has a third amino acid sequence, which binds specifically to an antigen, and the use of which in the defined test system for detection of the analyte brings about an analyte-specific detection reaction, and
d) a further antibody variant which has a fourth amino acid sequence and the antigen-binding capacity of which is greatly reduced compared to the further antigen-specific antibody such that its additional use in the defined test system for detection of the analyte reduces the analyte-specific detection reaction by a maximum of 15%,
and wherein the amino acid sequence of the further antibody variant, with the exception of one to three modified amino acid residues, is identical to the amino acid sequence of the further antigen-specific antibody.

10. The kit as claimed in claim 1, wherein the first antigen-specific antibody binds specifically to GPIb protein.

11. The kit as claimed in claim 10, wherein the first GPIb protein-specific antibody has, in the complementarity-determining region CDR-H3 of the heavy chain, the amino acid sequence according to SEQ ID NO. 1.

12. The kit as claimed in claim 11, wherein the amino acid sequence of the antibody variant, with the exception of two modified amino acid residues, is identical to the amino acid sequence of the first GPIb protein-specific antibody and has, in the complementarity-determining region CDR-H3 of the heavy chain, the amino acid sequence according to SEQ ID NO. 2.

13. A method for detecting an analyte in a body fluid sample, the method comprising:
a) providing a reaction mixture by mixing the sample with
i. a first antigen-specific antibody which has a first amino acid sequence, which binds specifically to an antigen, and the use of which in a defined test system for detection of the analyte brings about an analyte-specific detection reaction, and
ii. an antibody variant which has a second amino acid sequence and the antigen-binding capacity of which is greatly reduced compared to the first antibody such that its additional use in the defined test system for detection of the analyte reduces the analyte-specific detection reaction by a maximum of 15%; and
b) measuring a measurement variable in the reaction mixture, the measurement variable being influenced by the formation of a complex of antigen and the first antigen-specific antibody and correlating with the amount of analyte,
wherein the amino acid sequence of the antibody variant, with the exception of one to three modified amino acid residues, is identical to the amino acid sequence of the first antigen-specific antibody.

14. The method as claimed in claim 13, wherein the sample is first mixed with the antibody variant and the mixture thus produced is incubated, and the first antigen-specific antibody is then added to the mixture.

15. The method as claimed in claim 13, wherein the first antigen-specific antibody is an analyte-specific antibody and wherein the measurement variable measured is influenced by the formation of a complex of analyte and the first analyte-specific antibody.

16. The method as claimed in claim 13, wherein the first antigen-specific antibody is an antibody having specificity for a binding partner of the analyte, and wherein the measurement variable measured is influenced by the formation of a complex of analyte and the binding partner of the analyte and the first antigen-specific antibody having specificity for the binding partner of the analyte.

17. The method as claimed in claim 16, wherein the analyte is von Willebrand factor, and wherein the first antigen-specific antibody is an antibody having specificity for GPIb protein, and wherein the measurement variable measured is influenced by the formation of a complex of von Willebrand factor and the GPIb protein and the first antigen-specific antibody having specificity for GPIb protein.

18. The method as claimed in claim 13, wherein the first antigen-specific antibody is associated with a particulate solid phase and the agglutination of the particulate solid phase in the reaction mixture, which is influenced by the formation of a complex of antigen and the first antigen-specific antibody and which correlates with the amount of analyte, is measured.

19. The method as claimed in claim 13, wherein the method is interference-free detection of the analyte and the body fluid sample comprises interfering antibodies.

20. The method as claimed in claim 19, wherein the interfering antibodies are heterophilic antibodies and/or autoantibodies.


SEQUENCE LISTING (4 sequences)

SEQ ID NO: 1 - protein, 12 aa, Mus musculus
DTMIKGHYVMDY

SEQ ID NO: 2 - protein, 12 aa, unidentified
DTMIKGHSVFDY

SEQ ID NO: 3 - protein, 120 aa, Mus musculus
QVQLKESGPGLVAPSQSLSITCTVSGFSLTDYGVNWVRQPPGKGLEWLGMIWVDGSTDYNSALKSRLSISKDNSKSQVFLKMNSLQTDDTARYYCARDTMIKGHYVMDYWGQGTSVTVSS

SEQ ID NO: 4 - protein, 120 aa, unidentified
QVQLKESGPGLVAPSQSLSITCTVSGFSLTDYGVNWVRQPPGKGLEWLGMIWVDGSTDYNSALKSRLSISKDNSKSQVFLKMNSLQTDDTARYYCARDTMIKGHSVFDYWGQGTSVTVSS